(19) United States
(12) Patent Application Publication
(10) Pub. No.: US 2024/0125748 A1
(43) Pub. Date: Apr. 18, 2024

(54) GAS IDENTIFICATION METHOD AND GAS IDENTIFICATION SYSTEM

(71) Applicant: Panasonic Intellectual Property Management Co., Ltd., Osaka (JP)

(72) Inventors: Takuya HAYASHI, Osaka (JP); Yasuhiro MAMIYA, Osaka (JP)

(21) Appl. No.: 18/278,802

(22) PCT Filed: Mar. 8, 2022
(86) PCT No.: PCT/JP2022/009944

§ 371 Date: Aug. 24, 2023

(30) Foreign Application Priority Data
Mar. 12, 2021 (JP) 2021-040843

(51) Int. Cl. G01N 33/00

(52) U.S. Cl. CPC G01N 33/0034

(57) ABSTRACT

A gas identification method uses a sensor for outputting a signal corresponding to an adsorption concentration of a gas, and includes: acquiring a signal output from the sensor that is exposed to a sample gas only during a second period out of a measurement period including a first period, the second period following the first period, and a third period following the second period; extracting one or more feature quantities corresponding to the drift of the signal acquired; and, identifying the sample gas based on the one or more feature quantities extracted, using a learned logical model for identifying the sample gas, and outputting an identification result.

### CROSS-REFERENCE OF RELATED APPLICATIONS

This application is the U.S. National Phase under 35 U.S.C. § 371 of International Patent Application No. PCT/JP2022/009944, filed on Mar. 8, 2022, which in turn claims the benefit of Japanese Patent Application No. 2021-040843, filed on Mar. 12, 2021, the entire disclosures of which applications are incorporated by reference herein.

### TECHNICAL FIELD

The present disclosure relates to a gas identification method and a gas identification system.

### BACKGROUND ART

In gas identification, for example, a gas is identified based on a signal acquired from a sensor that is exposed to the gas. Patent Literature (PTL) 1 discloses, as a method for identifying an analyte using data from a pulse signal in which the analyte has been detected, a method using the intensity, wavelength, intensity ratio, kurtosis, and the like of the pulse signal as feature quantities.

### CITATION LIST

### Patent Literature

[PTL 1] WO 2018/207524

### SUMMARY OF INVENTION

### Technical Problem

When a sensor is used to identify a sample gas containing a chemical substance such as a volatile organic compound, reducing false determination is required.

Therefore, the present disclosure provides a gas identification method and the like capable of improving identification accuracy.

### Solution to Problem

A gas identification method according to one aspect of the present disclosure is a gas identification method using a sensor that outputs a signal corresponding to an adsorption concentration of a gas, the gas identification method including: acquiring a signal output from the sensor that is exposed to a sample gas only during a second period out of a measurement period including a first period, the second period following the first period, and a third period following the second period; extracting one or more feature quantities corresponding to a drift of the signal acquired; and identifying the sample gas based on the one or more feature quantities extracted, using a learned logical model for identifying the sample gas, and outputting an identification result.

A gas identification system according to one aspect of the present disclosure is a gas identification system including: a sensor that outputs a signal corresponding to an adsorption concentration of a gas; an exposure unit that exposes the sensor to a sample gas only during a second period out of a measurement period including a first period, the second period following the first period, and a third period following the second period; an acquisition circuit that acquires a signal output from the sensor in the measurement period; an extraction circuit that extracts one or more feature quantities corresponding to a drift of the signal acquired, a memory that stores a learned logical model for identifying the sample gas; and an identification circuit that identifies the sample gas based on the one or more feature quantities extracted, using the learned logical model, and outputs an identification result.

### Advantageous Effects of Invention

The gas identification method and the like according to one aspect of the present disclosure can improve identification accuracy.

### BRIEF DESCRIPTION OF DRAWINGS

FIG. 1 is a block diagram illustrating a schematic configuration of a gas identification system according to an embodiment.

FIG. 2 is a schematic diagram illustrating an example of a configuration of an exposure unit according to the embodiment.

FIG. 3 is a block diagram illustrating a schematic configuration of a gas identification system according to a variation of the embodiment.

FIG. 4 is a flowchart for explaining the operation of the gas identification system according to the embodiment.

FIG. 5 is a diagram illustrating an example of a control signal to the exposure unit and a signal output from a sensor according to the embodiment.

FIG. 6 is a diagram illustrating an example of a signal output from the sensor during a plurality of consecutive measurement periods according to the embodiment.

FIG. 7 is a diagram for explaining signal values acquired by an extraction circuit according to the embodiment.

FIG. 8 is another diagram for explaining signal values acquired by the extraction circuit according to the embodiment.

FIG. 9 is a diagram for explaining signal values acquired by the extraction circuit in a plurality of consecutive measurement periods according to the embodiment.

FIG. 10 is another diagram for explaining signal values acquired by the extraction circuit in the plurality of consecutive measurement periods according to the embodiment.

FIG. 11 is a diagram for explaining the rate of change and amount of change in the value of the signal output from the sensor according to the embodiment.

### DESCRIPTION OF EMBODIMENT

### (Circumstances Leading to the Present Disclosure)

In a case where a sensor that outputs a signal corresponding to the adsorption concentration of a gas is used for gas identification, for example, a chemical substance contained in a sample gas, such as a volatile organic compound, is identified as an identification target substance based on a feature quantity obtained using a signal output from the sensor when the sensor is exposed to the sample gas containing the chemical substance. For example, the signal output from the sensor changes due to the difference in the adsorption concentration onto the sensor depending on the type of the chemical substance. Therefore, the chemical substance contained in the sample gas is identified, for example, using the amount of change and the rate of change in the signal output from the sensor during a period when the sensor is exposed to the sample gas, and the like, as feature quantities. However, depending on the type of the chemical substance, the change in the signal associated with the adsorption of the chemical substance onto the sensor in the period may be similar among a plurality of chemical substances, resulting in false determination in identification. Hence the gas identification method is required to improve identification accuracy.

Meanwhile, in the gas identification, a period during which the sensor is not exposed to the sample gas is often set before or after the period during which the sensor is exposed to the sample gas. The inventors of the present invention have found that in a period after the exposure of the sensor to the sample gas, the value of the signal, which has changed due to the exposure of the sample gas, is difficult to revert to its original value, and the signal output from the sensor may drift. The present inventors have also found that such drift can vary even when the change in the signal associated with the adsorption of the chemical substance onto the sensor is similar. Based on such knowledge, the present disclosure provides a gas identification method and the like capable of improving identification accuracy.

### (Overview of the Present Disclosure)

An overview of one aspect of the present disclosure is as follows.

A gas identification method according to one aspect of the present disclosure is a gas identification method using a sensor that outputs a signal corresponding to an adsorption concentration of a gas, the gas identification method including: acquiring a signal output from the sensor that is exposed to a sample gas only during a second period out of a measurement period including a first period, the second period following the first period, and a third period following the second period; extracting one or more feature quantities corresponding to a drift of the signal acquired; and identifying the sample gas based on the one or more feature quantities extracted, using a learned logical model for identifying the sample gas, and outputting an identification result.

Thereby, in the extracting, one or more feature quantities corresponding to the drift of the signal are extracted, where the extracted feature quantity is different from the feature quantity corresponding to the output of the signal during the second period when the sensor is exposed to the sample gas, that is, the output depending on the adsorption concentration of the gas onto the sensor. Therefore, in the identifying, identification based on the drift of the signal is performed, and even in the case of identifying sample gases with similar outputs from the sensor, which depend on the adsorption concentrations of the gases, the sample gases can be identified with high identification accuracy.

For example, the gas identification may further include exposing the sensor to the sample gas only during the second period out of the measurement period. In the acquiring, the signal output from the sensor exposed in the exposing may be acquired.

Thereby, one or more feature quantities can be extracted using the signal output from the sensor exposed in the exposing.

For example, in the exposing, the sensor may be exposed to a reference gas during each of the first period and the third period.

Thereby, with the sensor being exposed to the reference gas as a reference for measurement during the first and third periods, even when the surrounding environment changes, a signal serving as a reference can be obtained from the sensor.

For example, in the acquiring, the signal output from the sensor may be acquired via a network.

Thereby, an output from a sensor measured at another location can be acquired.

For example, in the extracting: a first value may be acquired, the first value being a value of the signal when the value of the signal which has changed due to exposure of the sensor to the sample gas during the second period is reverting to a reference value during the third period; and at least one feature quantity among the one or more feature quantities may be extracted by using the first value acquired.

Thereby, the first value, which is the value of the signal when the value of the signal is reverting to the reference value directly related to the drift, can be used for the feature quantity extraction.

For example, in the acquiring, the signal output from the sensor may be acquired during a plurality of consecutive measurement periods each of which is the measurement period. In the extracting: first values are acquired in two or more measurement periods among the plurality of consecutive measurement periods, the first values each being the first value; and a difference between the first values acquired may be extracted as the at least one feature quantity among the one or more feature quantities.

Hence the signal is acquired during the plurality of consecutive measurement periods, whereby the drift of the acquired signal is likely to be large. Therefore, the difference between the first values in the plurality of respective measurement periods is likely to be large, and the difference in the extracted feature quantity is likely to be large according to the type of the sample gas, thus enabling further improvement in identification accuracy.

For example, in the acquiring, the signal output from the sensor may be acquired during a plurality of consecutive measurement periods each of which is the measurement period. In the extracting: first values may be acquired in two or more measurement periods among the plurality of consecutive measurement periods, the first values each being the first value; an approximate equation may be derived by using the first values acquired; and a coefficient of the approximate equation derived may be extracted as the at least one feature quantity among the one or more feature quantities.

Hence the signal is acquired during the plurality of consecutive measurement periods, whereby the drift of the acquired signal is likely to be large. In addition, by extracting the coefficient of the approximate equation obtained using the first value in each of the plurality of measurement periods as a feature quantity, it is possible to extract a feature quantity in which the variation of the first value is leveled.

For example, in the extracting: a second value may be acquired, the second value being a value of the signal at an end of the first period; and a difference between the first value acquired and the second value acquired may be extracted as the at least one feature quantity among the one or more feature quantities.

Thereby, a feature quantity corresponding to the drift can be extracted using the second value as a reference value.

For example, in the extracting, a value of the signal acquired at the end of the third period may be acquired as the first value.

It is thereby possible to use, as the first value, the value of the signal acquired at the end of the third period, which is reverting to the reference value during the third period and is likely to be stable.

For example, in the acquiring, the signal output from the sensor may be acquired during a plurality of consecutive measurement periods each of which is the measurement period. In the extracting: third values may be acquired in two or more measurement periods among the plurality of consecutive measurement periods, the third values each being a third value that is a value of the signal when the value of the signal changes due to exposure of the sensor to the sample gas during the second period; and a difference between the third values acquired may be extracted as at least one feature quantity among the one or more feature quantities.

Hence the signal is acquired during the plurality of consecutive measurement periods, whereby the drift of the acquired signal is likely to be large. Therefore, the difference between the third values in the plurality of respective measurement periods is likely to be large, and the difference in the extracted feature quantity is likely to be large according to the type of the sample gas, thus enabling further improvement in identification accuracy.

For example, in the acquiring, the signal output from the sensor may be acquired during a plurality of consecutive measurement periods each of which is the measurement period. In the extracting: a third value may be acquired in two or more measurement periods among the plurality of consecutive measurement periods, third values may be acquired in two or more measurement periods among the plurality of consecutive measurement periods, the third values each being a third value that is a value of the signal when the value of the signal changes due to exposure of the sensor to the sample gas during the second period; and a coefficient of the approximate equation derived may be extracted as at least one feature quantity among the one or more feature quantities.

Hence the signal is acquired during the plurality of consecutive measurement periods, whereby the drift of the acquired signal is likely to be large. In addition, by extracting the coefficient of the approximate equation obtained using the third value in each of the plurality of measurement periods as a feature quantity, it is possible to extract a feature quantity in which the variation of the third value is leveled.

For example, in the acquiring, the signal output from the sensor may be acquired during a plurality of consecutive measurement periods each of which is the measurement period. In the extracting, at least one feature quantity among the one or more feature quantities may be extracted based on the signal acquired during a second or subsequent measurement period in the plurality of measurement periods.

Thereby, the feature quantity is extracted using the signal output during the second and subsequent measurement periods when the drift is likely to be large, in the signal acquired during the plurality of consecutive measurement periods. Therefore, the difference in the extracted feature quantity is likely to be large according to the type of the sample gas, thus enabling further improvement in identification accuracy.

A gas identification system according to one aspect of the present disclosure is a gas identification system including: a sensor that outputs a signal corresponding to an adsorption concentration of a gas; an exposure unit that exposes the sensor to a sample gas only during a second period out of a measurement period including a first period, the second period following the first period, and a third period following the second period; an acquisition circuit that acquires a signal output from the sensor in the measurement period; an extraction circuit that extracts one or more feature quantities corresponding to a drift of the signal acquired, a memory that stores a learned logical model for identifying the sample gas; and an identification circuit that identifies the sample gas based on the one or more feature quantities extracted, using the learned logical model, and outputs an identification result.

Thereby, the extraction circuit extracts one or more feature quantities corresponding to the drift of the signal, where the extracted feature quantity is different from the feature quantity corresponding to the output of the signal during the second period when the sensor is exposed to the sample gas, that is, the output depending on the adsorption concentration of the gas onto the sensor. Therefore, in the identification circuit, identification based on the drift of the signal is performed, and even in the case of identifying sample gases with similar outputs from the sensor, which depend on the adsorption concentrations of the gases, the sample gases can be identified with high identification accuracy.

Hereinafter, an exemplary embodiment will be described in detail with reference to the drawings as appropriate. Note that the embodiment described below shows a comprehensive or a specific example. Numerical values, shapes, materials, components, arrangement and connection modes of the components, steps, the order of the steps, and the like, which will be shown in the following embodiment, are only examples and are not intended to limit the present disclosure. Among the components in the following embodiment, components not recited in independent claims are described as optional components.

In the present specification, a term indicating a relationship between elements, such as parallel, a term indicating the shape of an element, and a numerical range are not expressions expressing only strict meanings but expressions meaning to include substantially equivalent ranges, for example, differences of about a few percent.

Each of the drawings is not necessarily strictly illustrated. In the drawings, substantially the same components are denoted by the same reference numerals, and duplicated description is omitted or simplified.

In the present specification, ordinal numbers such as “first” and “second” do not mean the number or order of steps, components, or the like unless otherwise specified, and are used to avoid confusion and to distinguish between steps, components of the same type, and the like.

### Embodiment

### [Configuration]

First, a configuration of a gas identification system according to an embodiment will be described.

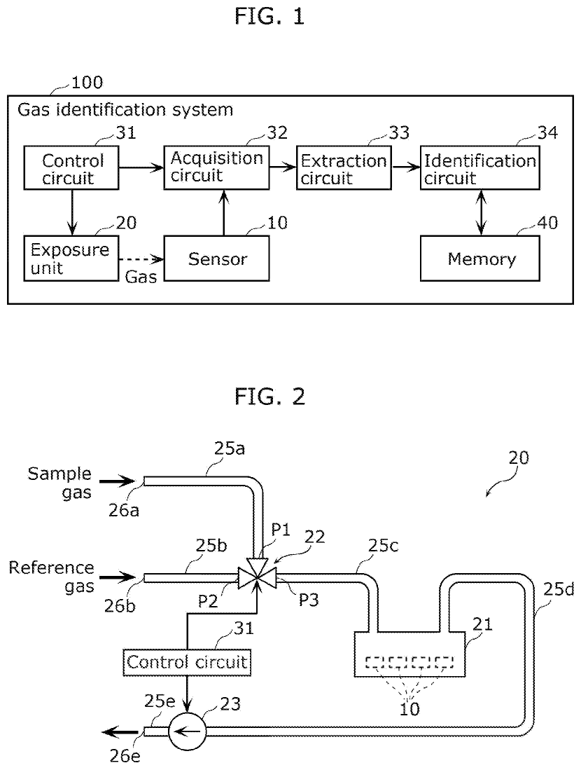

FIG. 1 is a block diagram illustrating a schematic configuration of gas identification system 100 according to the present embodiment.

As illustrated in FIG. 1, gas identification system 100 according to the present embodiment includes sensor 10, exposure unit 20, control circuit 31, acquisition circuit 32, extraction circuit 33, identification circuit 34, and memory 40. Gas identification system 100 identifies a sample gas based on the output of sensor 10 exposed to the sample gas. The sample gas contains, for example, a chemical substance to be identified. The sample gas is, for example, a gas collected from food, exhaled air collected from a human body, air around a human body, or air collected from a room in a building.

Gas identification system 100 identifies, for example, a chemical substance contained in the sample gas. Specifically, gas identification system 100 identifies which of a plurality of identification target substances is contained in the sample gas as a chemical substance. Gas identification system 100 may identify whether or not the identification target substance is contained in the sample gas.

The identification target substance is, for example, a volatile organic compound, but may be an inorganic gas such as ammonia or carbon monoxide. Gas identification system 100 is used, for example, to identify odors. In this case, the volatile organic compound is, for example, a molecule serving as an odor component.

Sensor 10 is a sensor that outputs a signal corresponding to the adsorption concentration of the gas. Sensor 10 is, for example, an electrochemical sensor, a semiconductor sensor, a field-effect transistor sensor, a surface acoustic wave sensor, a quartz crystal sensor, a resistance change sensor, or the like.

Sensor 10 includes, for example, a sensing unit and a pair of electrodes electrically connected to the sensing unit. In the sensing unit, for example, an electrical resistance value changes according to the adsorption concentration of the gas. A signal corresponding to the electrical resistance value of the sensing unit of sensor 10 is acquired by acquisition circuit 32 as a voltage signal or a current signal via the pair of electrodes, for example.

The sensing unit is formed of, for example, a resin material that is an adsorbent for absorbing gas, and conductive particles dispersed in the resin material. Examples of the resin material include, for example, polyalkylene glycol resin, polyester resin, and silicone resin. The resin material is, for example, a material commercially available as a stationary phase in a column of gas chromatography. From the viewpoint of durability and gas adsorption, the resin material may be, for example, a silicone resin with various substituents such as phenyl and methyl groups on the side chain, which is commercially available as the stationary phase of the column. The sensing unit is not limited to being formed of the resin material and the conductive particles, but may be any member with its electrical resistance value changing due to the adsorption of the gas. The sensing unit may be formed of an inorganic material such as metal oxide, or porous ceramics, for example.

Gas identification system 100 includes a plurality of sensors 10, for example. The sensing units (specifically, the resin materials constituting the sensing units) in at least two respective sensor units 10 among the plurality of sensors 10 are formed of mutually different types of materials, for example. The types of materials of the respective sensing units in all the plurality of sensors 10 may be different from each other. Different types of materials exhibit different adsorption behaviors for the same chemical substance. Thus, the plurality of sensors 10 output different signals for the same chemical substance. This enables extraction of different feature quantities from the outputs of the plurality of respective sensors 10, thereby improving the identification accuracy in gas identification system 100.

Exposure unit 20 is an exposure mechanism that exposes sensor 10 to the gas based on the control of control circuit 31. Specifically, exposure unit 20 exposes sensor 10 to the sample gas only during a second period out of a measurement period made up of a first period, the second period following the first period, and a third period following the second period. Exposure unit 20 may expose sensor 10 to the reference gas during the first and third periods. The reference gas is a gas that serves as a reference for measurement, for example, a gas that does not contain an identification target substance. In addition, the reference gas is, for example, a gas that is less easily adsorbed onto the sensing unit of sensor 10 than the identification target substance. Specific examples of the reference gas include inert gases such as air and nitrogen, and a gas obtained by removing a chemical substance from the sample gas with a filter or the like. By outputting the signal from sensor 10 exposed to the reference gas during the first and third periods as described above, even when the surrounding environment changes or in other cases, a signal serving as a reference corresponding to the surrounding environment for each measurement can be obtained. Therefore, using such a signal can lead to improvement in identification accuracy to be described later.

The specific configuration of exposure unit 20 will now be described. FIG. 2 is a schematic diagram illustrating an example of the configuration of exposure unit 20 according to the present embodiment. As illustrated in FIG. 2, exposure unit 20 has, for example, housing 21, three-way solenoid valve 22, intake pump 23, and a plurality of pipes 25a, 25b, 25c, 25d, 25e.

Intake port 26a for introducing the sample gas is provided at one end of pipe 25a. Intake port 26a is provided, for example, in a space filled with the sample gas. Intake port 26b for introducing the reference gas is provided at one end of pipe 25b. Intake port 26b is provided, for example, in a space filled with the reference gas. Exhaust port 26e for discharging the introduced sample gas and reference gas are provided at one end of pipe 25e.

Housing 21 is a box-shaped container that houses sensor 10. Inside housing 21, for example, the plurality of sensors 10 are arranged in an array. One end of each of pipe 25c and pipe 25d is connected to housing 21. By the operation of intake pump 23 to be described later, the gas flows from one end of pipe 25c to one end of pipe 25d. The plurality of sensors 10 are placed in a flow path through which the gas flows.

The sample gas introduced from intake port 26a is introduced into the interior of housing 21 via pipe 25a, three-way solenoid valve 22, and pipe 25c. The reference gas introduced from intake port 26b is introduced into the interior of housing 21 via pipe 25b, three-way solenoid valve 22, and pipe 25c. The sample gas and the reference gas introduced into the interior of housing 21 are discharged from exhaust port 26e via pipe 25d, intake pump 23, and pipe 25e.

Three-way solenoid valve 22 is a solenoid valve for switching the gas introduced into housing 21. Three-way solenoid valve 22 has input port P1 to which the other end of pipe 25a is connected, input port P2 to which the other end of pipe 25b is connected, and output port P3 to which the other end of pipe 25c is connected. The opening and closing of each port in three-way solenoid valve 22 are controlled under the control of control circuit 31. Three-way solenoid valve 22 switches between a first state in which input port P1 and output port P3 are electrically connected and a second state in which input port P2 and output port P3 are electrically connected under the control of control circuit 31. In the first state, input port P1 and output port P3 are open, and input port P2 is closed. In the second state, input port P2 and output port P3 are open, and input port P1 is closed.

Intake pump 23 is a pump for introducing the sample gas and the reference gas into the interior of housing 21 and discharging the introduced sample gas and reference gas from exhaust port 26e. The operation of intake pump 23 is controlled under the control of control circuit 31. The intake port of intake pump 23 is connected to the other end of pipe 25d. The exhaust port of intake pump 23 is connected to the other end of pipe 25e.

With such a configuration, the sample gas is introduced into the interior of housing 21 when intake pump 23 is operating and three-way solenoid valve 22 is in the first state. Thereby, exposure unit 20 exposes the plurality of sensors 10 to the sample gas. When intake pump 23 is operating and three-way solenoid valve 22 is in the second state, the reference gas is introduced into the interior of housing 21. Thereby, exposure unit 20 exposes the plurality of sensors 10 to the reference gas.

Note that the configuration of exposure unit 20 is not limited to the configuration illustrated in FIG. 2, and is not particularly limited as long as sensor 10 can be exposed to the sample gas. For example, exposure unit 20 may be configured so that the sample gas and the reference gas are introduced into the interior of housing 21 through separate pipes, bypassing three-way solenoid valve 22. Exposure unit 20 may not include intake pump 23 and may be configured to allow carrier gas to constantly flow into housing 21 to mix the sample gas in the carrier gas. The reference gas may not be introduced, and housing 21 may be vacuumed by intake pump 23 after sensor 10 is exposed to the sample gas. Exposure unit 20 may further include various removal filters for removing moisture, particulates, and the like from the sample gas and the reference gas, an electromagnetic control valve for adjusting the flow rate of each pipe, a check valve for preventing backflow of each pipe, and the like.

Referring again to FIG. 1, control circuit 31 controls the operation of exposure unit 20, specifically the operations of three-way solenoid valve 22 and intake pump 23, as described above. Control circuit 31 may output, to acquisition circuit 32, information indicating the timing of the operation of exposure unit 20.

Acquisition circuit 32 acquires a signal output from sensor 10 during the measurement period. Acquisition circuit 32 acquires, for example, a voltage signal or a current signal as a signal output corresponding to the electrical resistance value of the sensing unit of sensor 10.

Extraction circuit 33 extracts one or more feature quantities corresponding to the drift of the signal acquired by acquisition circuit 32. Extraction circuit 33 may extract a feature quantity other than the feature quantity corresponding to the drift, from the signal acquired by acquisition circuit 32. When a plurality of sensors 10 are provided, extraction circuit 33 extracts one or more feature quantities from the signal output from each of the plurality of sensors 10.

Identification circuit 34 identifies a sample gas based on one or more feature quantities extracted by extraction circuit 33, using a learned logical model. Identification circuit 34 identifies, for example, which of a plurality of identification target substances is contained in the sample gas. Identification circuit 34 may identify whether or not the sample gas contains the identification target substance. Identification circuit 34 receives one or more feature quantities as an input and outputs an identification result. Identification circuit 34 outputs, for example, information for displaying the identification result on a display (not illustrated) or the like provided in the gas identification system. Identification circuit 34 may output the information indicating the identification result to memory 40 and store the information in memory 40. Identification circuit 34 may output information indicating the identification result to an external device.

Control circuit 31, acquisition circuit 32, extraction circuit 33, and identification circuit 34 are implemented by a microcomputer or a processor incorporating a program for performing the above processing. Control circuit 31, acquisition circuit 32, extraction circuit 33, and identification circuit 34 may each be implemented by a dedicated logic circuit that performs the above processing.

Memory 40 is a storage device that stores the learned logical model used in identification circuit 34. Memory 40 is implemented, for example, by a semiconductor memory.

The learned logical model is a logical model that identifies the sample gas. Specifically, the learned logical model is, for example, a logical model that identifies which of a plurality of identification target substances is contained in the sample gas. For example, the learned logical model receives as an input one or more feature quantities extracted by extraction circuit 33 and outputs which of the plurality of identification target substances is contained in the sample gas. The learned logical model may output whether or not the sample gas contains the identification target substance.

The learned logical model is constructed, for example, by performing machine learning with a known identification target substance and one or more feature quantities, extracted by extraction circuit 33 using the known identification target substance, as teacher data. The method used to construct the logical model in machine learning is not particularly limited. For example, a neural network is used to construct a logical model in machine learning. That is, the learned logical model includes, for example, a neural network. A random forest, a support vector machine, a self-organizing map, or the like may be used to construct a logical model in machine learning.

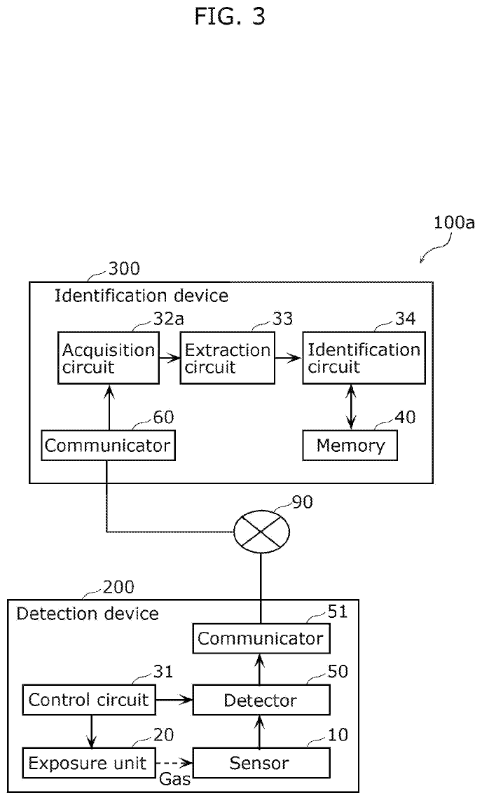

Note that gas identification system 100 is implemented, for example, as a single gas identification device including the above components, but may be implemented by a plurality of devices. When gas identification system 100 is implemented by a plurality of devices, the components included in gas identification system 100 may be distributed to the plurality of devices in any manner. Here, an example of the gas identification system implemented by a plurality of devices will be described with reference to FIG. 3. FIG. 3 is a block diagram illustrating a schematic configuration of gas identification system 100a according to a variation of the embodiment.

As illustrated in FIG. 3, gas identification system 100a includes detection device 200 and identification device 300.

Detection device 200 includes sensor 10, exposure unit 20, control circuit 31, detector 50, and communicator 51. Sensor 10, exposure unit 20, and control circuit 31 have the same configurations as those in gas identification system 100 described above, for example.

Detector 50 acquires a signal output from sensor 10 during the measurement period. For example, a voltage signal or a current signal is acquired as a signal corresponding to the electrical resistance value of the sensing unit of sensor 10. Further, information indicating the timing for controlling exposure unit 20 is acquired from control circuit 31. Detector 50 transmits the acquired signal and information to identification device 300 using communicator 51. Detector 50 is implemented by a microcomputer or a processor incorporating a program for performing the above processing. Detector 50 may be implemented by a dedicated logic circuit that performs the above processing.

Communicator 51 is a communication module (communication circuit) for detection device 200 to communicate with identification device 300 via wide-area communication network 90 such as the Internet, which is an example of a network. Communicator 51 may perform wired or wireless communication. The communication standard used for the communication performed by communicator 51 is not particularly limited.

Identification device 300 includes acquisition circuit 32a, extraction circuit 33, identification circuit 34, memory 40, and communicator 60. Extraction circuit 33, identification circuit 34, and memory 40 have the same configurations as those in gas identification system 100 described above, for example.

Acquisition circuit 32a acquires the signal output from sensor 10 during the measurement period and acquired by detector 50, via wide-area communication network 90. Acquisition circuit 32a communicates with detection device 200 via wide-area communication network 90 using communicator 60.

Communicator 60 is a communication module (communication circuit) for identification device 300 to communicate with detection device 200 via wide-area communication network 90. Communicator 60 may perform wired or wireless communication. The communication standard used for the communication performed by communicator 60 is not particularly limited.

### [Operation]

Next, the operation of the gas identification system according to the present embodiment will be described. In the following, the operation of gas identification system 100 will be mainly described, but a similar operation is performed for gas identification system 100a unless otherwise specified.

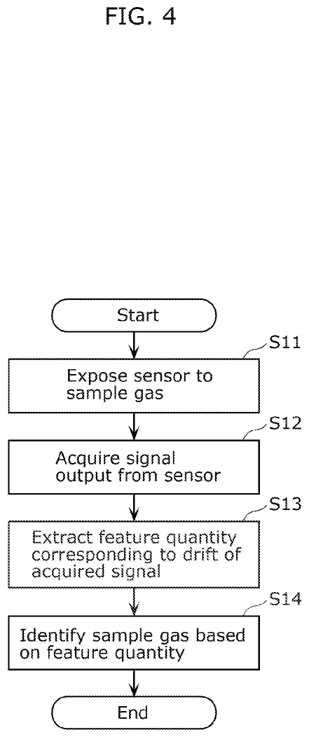

FIG. 4 is a flowchart illustrating the operation of gas identification system 100 according to the present embodiment. In other words, FIG. 4 is a flowchart of a gas identification method performed by gas identification system 100. The gas identification method according to the present embodiment includes an exposure step, an acquisition step, an extraction step, and an identification step.

In the present specification, the exposure step is an example of a fourth step, the acquisition step is an example of a first step, the extraction step is an example of a second step, and the identification step is an example of a third step.

### (1) Exposure Step and Acquisition Step

As illustrated in FIG. 4, first, in the exposure step, exposure unit 20 exposes sensor 10 to the sample gas only during the second period out of the measurement period (step S11). Exposure unit 20 exposes sensor 10 to the reference gas during the first and third periods. For example, control circuit 31 operates intake pump 23 to control the opening and closing of each port of three-way solenoid valve 22, thereby exposing sensor 10 to the reference gas during the first and third periods and exposing sensor 10 to the sample gas during the second period. In step S11, for example, in a plurality of consecutive measurement periods, sensor 10 is exposed to the sample gas only during the second period out of the measurement period.

In the acquisition step, acquisition circuit 32 acquires the signal output from sensor 10 exposed in step S11 (step S12). That is, acquisition circuit 32 acquires the signal output from sensor 10 exposed to the sample gas only during the second period out of the measurement period.

In gas identification system 100a, detector 50 acquires the signal output from sensor 10 exposed in step S11. Acquisition circuit 32a acquires from detector 50 the signal output from sensor 10 exposed in step S11 via wide-area communication network 90. This enables acquisition circuit 32a to acquire the signal output from sensor 10 even when sensor 10 is located away from identification device 300.

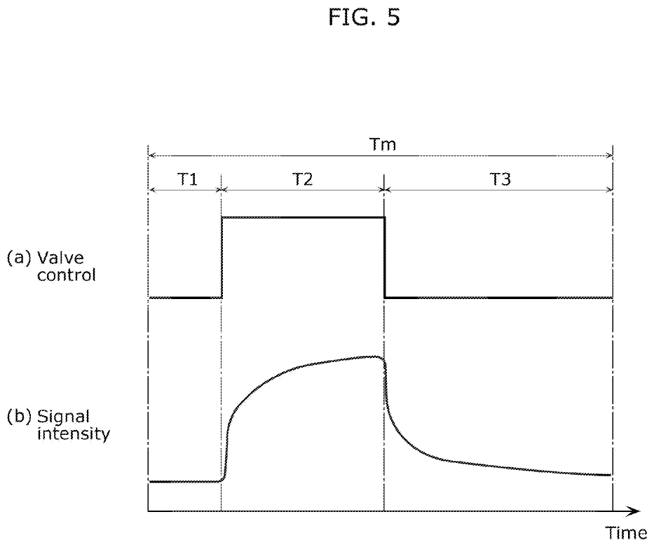

FIG. 5 illustrates an example of a control signal to exposure unit 20 and a signal output from sensor 10. FIG. 5 illustrates a control signal to three-way solenoid valve 22 and the intensity of a signal output from sensor 10 during measurement period Tm made up of first period T1, second period T2, and third period T3. (a) of FIG. 5 is a graph illustrating an example of a temporal change in the control signal output from control circuit 31. In (a) of FIG. 5, three-way solenoid valve 22 is controlled to be in the first state when the control signal is at the high level, and three-way solenoid valve 22 is controlled to be in the second state when the control signal is at the low level. (b) of FIG. 5 illustrates an example of a temporal change in the intensity (e.g., voltage) of the signal output from sensor 10.

In step S11, for example, as illustrated in (a) of FIG. 5, during first period T1 and third period T3, three-way solenoid valve 22 is in the second state, and exposure unit 20 exposes sensor 10 to the reference gas. During second period T2, three-way solenoid valve 22 is in the first state, and exposure unit 20 exposes sensor 10 to the sample gas. As a result, the signal acquired in step S12 changes, for example, as illustrated in (b) of FIG. 5. First, during first period T1 when sensor 10 is exposed to the reference gas, the signal value hardly changes. Next, during second period T2 when sensor 10 is exposed to the sample gas, the signal value changes (e.g., increases) due to the adsorption of the sample gas (mainly the chemical substance contained in the sample gas) onto the sensing unit of sensor 10. During third period T3 when sensor 10 is again exposed to the reference gas, the sample gas (mainly the chemical substance contained in the sample gas) leaves the sensing unit of sensor 10, and the signal value that changed during the second period is reverting to a reference value. The reference value is, for example, a value before the signal value begins to change due to the exposure of sensor 10 to the sample gas.

The lengths of the first, second, and third periods are not particularly limited, and are set according to, for example, the type of sensor 10, the type of the identification target substance, and the like. The length of first period T1 is, for example, 1 second or more and 10 seconds or less. The length of second period T2 is, for example, 5 seconds or more and 30 seconds or less. The length of the third period is, for example, 10 seconds or more and 100 seconds or less.

In step S11, for example, in each of a plurality of consecutive measurement periods Tm, exposure unit 20 exposes sensor 10 to the sample gas only during the second period out of measurement period Tm. Then, in step S12, acquisition circuit 32 acquires the signal output from sensor 10 during the plurality of consecutive measurement periods Tm.

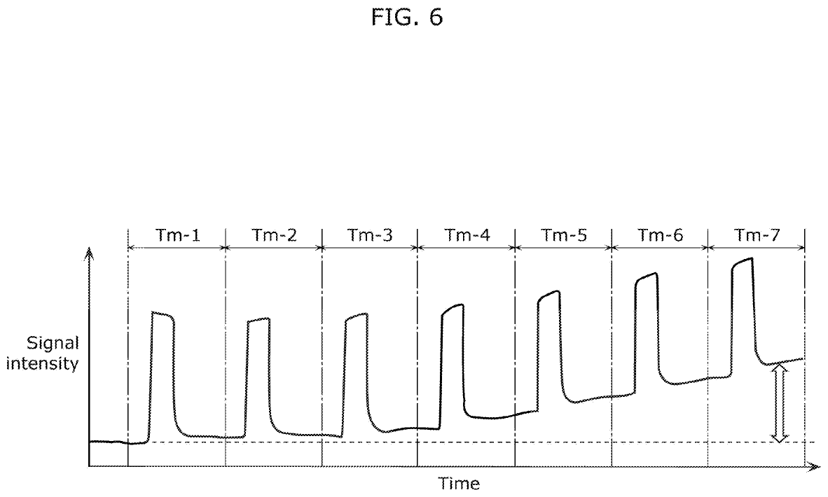

FIG. 6 illustrates an example of the signal output from sensor 10 during the plurality of consecutive measurement periods. Acquisition circuit 32 acquires the signal output from sensor 10, for example, during seven consecutive measurement periods Tm-1 to Tm-7, as illustrated in FIG. 6. In each of measurement periods Tm-1 to Tm-7, the operation described in FIG. 5 is performed, and the same operation is repeated. Thus, in second and subsequent measurement periods Tm-2 to Tm-7, next measurement period Tm starts before the signal value that changed in second period T2 reverts to the reference value, causing an increase in drift, which will be described in detail later. Note that the number of consecutive measurement periods Tm is not limited, and is set according to, for example, the type of sensor 10, the type of the identification target substance, and the like.

### (2) Extraction Step

Referring again to FIG. 4, next, in the extraction step, extraction circuit 33 extracts one or more feature quantities corresponding to the drift of the signal acquired in step S12 (step S13). Extraction circuit 33 extracts one or more feature quantities using the acquired signal values.

As illustrated in FIG. 6, the baseline of the signal output from sensor 10 has dissociated from the reference value to cause drift. Such drift of the signal is considered to be attributed, for example, to the difficulty for the gas to escape from the sensing unit of sensor 10, and the magnitude of the drift changes according to the combination of the sample gas (specifically, the chemical substance contained in the sample gas) and the material of the sensing unit of sensor 10. For example, when the sample gas is difficult to escape from the sensing unit of sensor 10, the value of the output from sensor 10 does not return to the reference value to cause larger drift. On the other hand, when the sample gas easily escapes from the sensing unit of sensor 10, the drift is smaller, or no drift occurs. Extraction circuit 33 extracts one or more feature quantities corresponding to the drift of the signal for use in the identification of the sample gas.

For example, extraction circuit 33 acquires at least one of the first, second, or third values as a signal value from the acquired signal and extracts one or more feature quantities using the acquired value.

First, the first, second and third values acquired in extraction circuit 33 will be described.

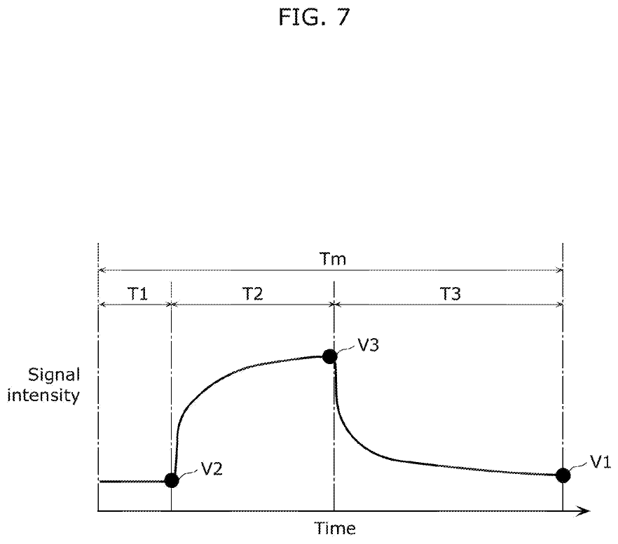

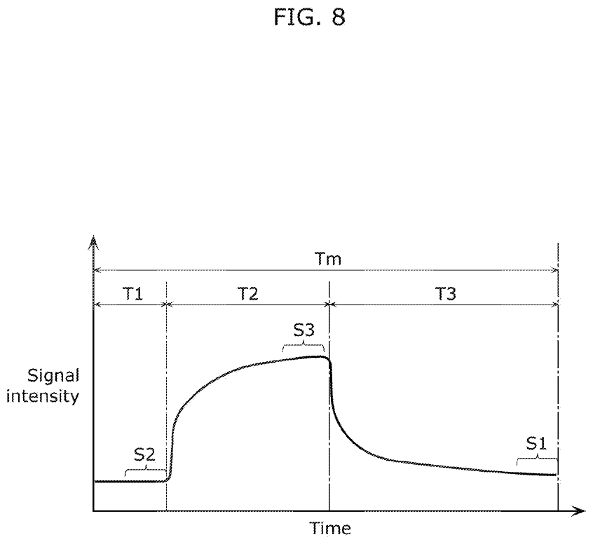

FIGS. 7 and 8 are diagrams for explaining signal values acquired by extraction circuit 33. FIGS. 7 and 8 each illustrate a temporal change in the intensity (e.g., voltage) of the signal output from sensor 10 acquired by acquisition circuit 32.

The first value is a signal value when the signal value that changed due to the exposure of sensor 10 to the sample gas in second period T2 is reverting to the reference value in third period T3. The first value is, for example, value V1 of the signal at a predetermined time point in third period T3, as illustrated in FIG. 7. The first value may be an average value of signal values in predetermined interval S1 in third period T3, as illustrated in FIG. 8. The length of interval S1 is, for example, 0.1 second or more and 5 seconds or less.

The first value may be a signal value acquired at the end of third period T3. In this case, the first value may be value V1 of the signal at the last time point in third period T3, or may be the average value of the values of the signal in interval S1 including the last time point in third period T3. This enables extraction circuit 33 to use the signal value that is stable during third period T3 as the first value. The first value may be value V1 of the signal at a time point after the lapse of a predetermined time from the start of third period T3, or may be the average value of the signal values in interval S1 starting after the lapse of a predetermined time from the start of third period T3. The predetermined time is, for example, at least half the length of third period T3.

As described above, the first value is the signal value in third period T3 during which sensor 10 is not exposed to the sample gas after second period T2 during which sensor 10 is exposed to the sample gas, so that the first value is less likely to be affected by the exposure to the sample gas and is suitable as a value indicating the change of the baseline in the signal.

The second value is a signal value acquired at the end of first period T1. The second value is, for example, value V2 of the signal at the last time point in first period T1, as illustrated in FIG. 7. The second value may be the average value of the signal values in interval S2 including the last in first period T1, as illustrated in FIG. 8. The length of interval S2 is, for example, 0.1 second or more and 5 seconds or less.

As described above, the second value is the signal value in first period T1 during which sensor 10 is not exposed to the sample gas before second period T2 during which sensor 10 is exposed to the sample gas, so that the second value is suitable as a value indicating the reference value for the signal.

The third value is a signal value when the signal value changes due to the exposure of sensor 10 to the sample gas in second period T2. The third value is, for example, value V3 of the signal at a predetermined time point in second period T2, as illustrated in FIG. 7. The third value may be the average value of the signal values in a predetermined interval S3 in second period T2, as illustrated in FIG. 8. The length of interval S3 is, for example, 0.1 second or more and 5 seconds or less.

The third value is, for example, a signal value at the timing when the signal value is maximum in second period T2. In this case, the third value may be value V3 of the signal at the time point at which the signal value is maximum in second period T2, or may be the average value of the signal values in interval S3 including the time point at which the signal value is maximum in second period T2. The third value may be value V3 of the signal at a time point after the lapse of a predetermined time from the start of second period T2, or may be the average value of the signal values in interval S3 starting after the lapse of a predetermined time from the start of second period T2. The predetermined time is, for example, a time more than half the length of second period T2.

The third value is a value at the time of its change due to the adsorption of the sample gas onto adsorption on sensor 10, and when the baseline of the signal deviates, the third value also deviates. Hence the feature quantity corresponding to the drift can be extracted using the third value.

Extraction circuit 33 acquires at least one of the first, second, or third values from the signal output from sensor 10 in two or more measurement periods among the plurality of consecutive measurement periods Tm-1 to Tm-7, for example. By acquiring the signal during the plurality of consecutive measurement periods Tm-1 to Tm-7 in this manner, the drift of the acquired signal is likely to be large, thus enabling improvement in identification accuracy to be described later.

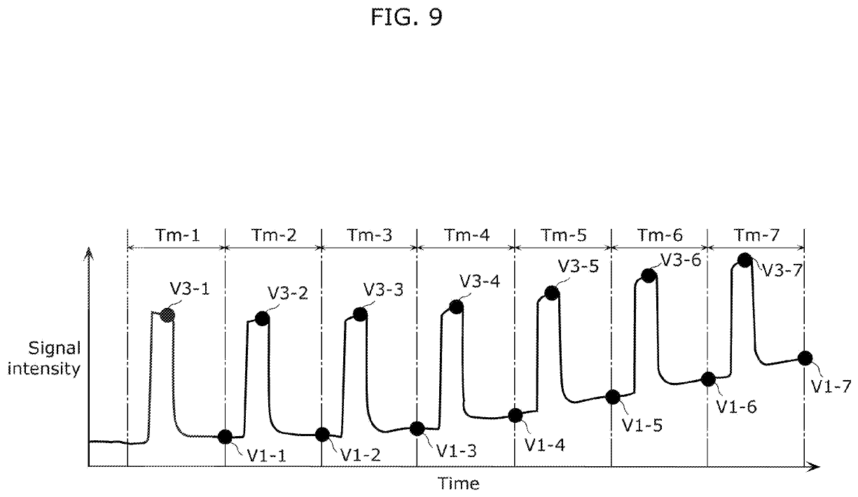

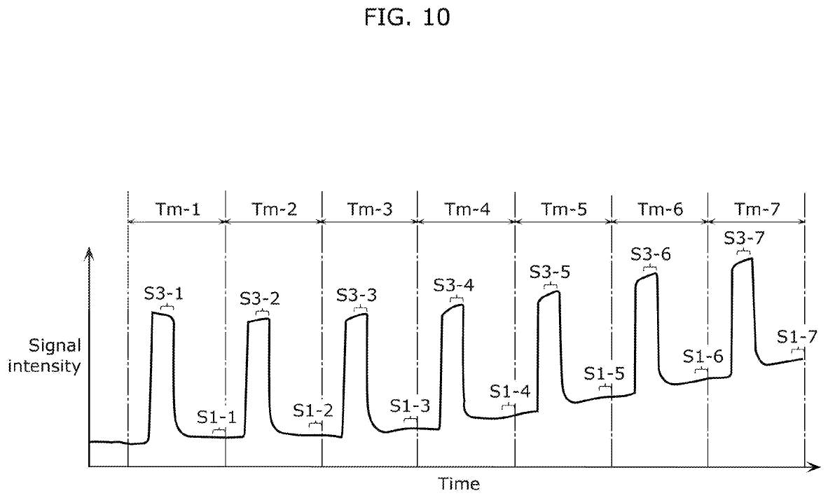

FIGS. 9 and 10 are diagrams for explaining the signal values acquired by extraction circuit 33 in the plurality of consecutive measurement periods Tm-1 to Tm-7. FIGS. 9 and 10 each illustrate the temporal change in the intensity (e.g., voltage) of the signal output from sensor 10 acquired by acquisition circuit 32.

As illustrated in FIGS. 9 and 10, when acquiring the first value, extraction circuit 33 acquires at least one of the respective first values in first measurement period Tm-1 to seventh measurement period Tm-7. The second and third values are acquired in the same manner as the case where the first value is acquired. When acquiring the first, second, or third value in the plurality of measurement periods Tm, extraction circuit 33 acquires the first, second, or third value, for example, at the same timing in each measurement period Tm.

Specifically, extraction circuit 33 acquires, for example, at least one of signal values V1-1 to V1-7 corresponding to signal value V1 illustrated in FIG. 7 as the first value. Alternatively, extraction circuit 33 acquires, for example, an average value of signal values in at least one of intervals S1-1 to S1-7 corresponding to interval S1 illustrated in FIG. 8 as the first value.

Extraction circuit 33 acquires, for example, at least one of signal values V3-1 to V3-7 corresponding to signal value V3 illustrated in FIG. 7 as the third value. Alternatively, extraction circuit 33 acquires, for example, an average value of signal values in at least one of intervals S3-1 to S3-7 corresponding to interval S3 illustrated in FIG. 8 as the third value. Although not illustrated in the drawing, the second value is acquired in the same manner as the first and third values.

Next, a specific example of a feature quantity extraction method by extraction circuit 33 will be described.

First, a first example of the feature quantity extraction method by extraction circuit 33 will be described. In the first example, for example, extraction circuit 33 acquires the first value in each of two or more measurement periods Tm among the plurality of consecutive measurement periods Tm-1 to Tm-7 illustrated in FIGS. 9 and 10, and extracts the difference between the acquired first values as a feature quantity. For example, extraction circuit 33 extracts, as a feature quantity, the difference between the first values in two respective measurement periods Tm separated from each other. Two measurement periods Tm separated from each other are, for example, measurement periods Tm-2 and Tm-7, which are second and last measurement periods Tm among the plurality of consecutive measurement periods Tm-1 to Tm-7. As a result, the difference between the first values is likely to be large, and identification accuracy, which will be described later, is likely to be high.

Extraction circuit 33 may extract the difference between the first values in two respective consecutive measurement periods Tm (e.g., measurement period Tm-2 and measurement period Tm-3) as a feature quantity.

Extraction circuit 33 may extract a plurality of feature quantities by changing the combination of two measurement periods Tm for taking the difference in the first values into a plurality of combinations. For example, extraction circuit 33 may extract the differences between the first values in all combinations of two consecutive measurement periods Tm as a plurality of feature quantities. When changing the combination of two measurement periods Tm into a plurality of combinations, extraction circuit 33 may extract each of the differences between the plurality of extracted first values as a feature quantity, or may extract the average value of the differences between the plurality of extracted first values as a feature quantity.

In the first example, extraction circuit 33 may acquire the third value instead of the first value. That is, extraction circuit 33 may acquire the third value in each of two or more measurement periods Tm among the plurality of consecutive measurement periods Tm-1 to Tm-7 illustrated in FIGS. 9 and 10, and extract the difference between the acquired third values as a feature quantity. This is explained by replacing the first value mentioned in the above description with the third value. Extraction circuit 33 may extract both the difference between the first values described above and the difference between the third values as feature quantities, respectively.

As described above, in the first example, extraction circuit 33 can extract the feature quantity corresponding to the drift that indicates a change in the baseline of the signal, by taking the difference between the values at the same timings in the plurality of respective measurement periods Tm. With the difference being taken between the first values or between the third values acquired across the plurality of measurement periods Tm, the extracted feature quantity is likely to be large.

Next, a second example of the feature quantity extraction method by extraction circuit 33 will be described. In the second example, extraction circuit 33 acquires the first and second values in measurement period Tm and extracts the difference between the acquired first and second values as a feature quantity. For example, extraction circuit 33 acquires the first and second values in at least one measurement period Tm among the plurality of consecutive measurement periods Tm-1 to Tm-7 illustrated in FIGS. 9 and 10, and extracts the difference between the first and second values acquired in the same measurement period Tm as a feature quantity. When extracting the difference between the first and second values in the plurality of measurement periods, extraction circuit 33 may extract each of the differences between the plurality of extracted first and second values as a feature quantity, or may extract the average value of the differences between the plurality of extracted first and second values as a feature quantity.

As described above, in the second example, extraction circuit 33 can extract the feature quantity corresponding to the drift of the signal by taking the difference of the second value serving as the reference value from the first value.

Next, a third example of the feature quantity extraction method by extraction circuit 33 will be described. In the third example, for example, extraction circuit 33 acquires the first value in each of two or more measurement periods Tm among the plurality of consecutive measurement periods Tm-1 to Tm-7 illustrated in FIGS. 9 and 10. Extraction circuit 33 acquires, for example, the first values in all respective measurement periods Tm of second measurement period Tm-2 and later among the plurality of consecutive measurement periods Tm-1 to Tm-7.

Within measurement periods Tm of second measurement period Tm-2 and later, there may be measurement period Tm in which extraction circuit 33 does not acquire the first value. That is, extraction circuit 33 may acquire the first value in one or more of measurement periods Tm of second measurement period Tm-2 and later among the plurality of consecutive measurement periods Tm-1 to Tm-7. For example, extraction circuit 33 may acquire the first values in all respective measurement periods Tm of third measurement period Tm-3 and later, or fourth measurement period Tm-4 and later, among the plurality of consecutive measurement periods Tm-1 to Tm-7.

Extraction circuit 33 may acquire the first values in all respective measurement periods Tm of the plurality of consecutive measurement periods Tm-1 to Tm-7.

Extraction circuit 33 then derives an approximate equation using the acquired first values and extracts the coefficient of the derived approximate equation as a feature quantity. The approximate equation is, for example, an approximate equation when the first value in each measurement period Tm is a function of time. The approximate equation is, for example, a linear (i.e., linear approximation) or quadratic expression. The approximate equation may be a polynomial other than a quadratic, and may be an exponential, logarithmic, or power expression. A known method can be used to derive the approximate equation. In the case of a linear approximate equation, for example, the approximate equation can be derived using the least squares method.

In the third example, extraction circuit 33 may acquire the third value instead of the first value. That is, extraction circuit 33 may acquire the third value in each of two or more measurement periods Tm among the plurality of consecutive measurement periods Tm-1 to Tm-7 illustrated in FIGS. 9 and 10, derive an approximate equation using the acquired third values, and extract the coefficient of the derived approximate equation as a feature quantity. This is explained by replacing the first value mentioned in the above description with the third value. Extraction circuit 33 may extract both the coefficient of the approximate equation using the first values described above and the coefficient of the approximate equation using the third values as feature quantities, respectively.

As described above, in the third example, an approximate equation corresponding to the baseline of the signal is derived, so that extraction circuit 33 can extract the feature quantity corresponding to the drift of the signal. By extracting the coefficient of the approximate equation obtained using the first or third value in each of the plurality of measurement periods Tm as the feature quantity, it is possible to extract a feature quantity in which the variation of the first or third value is leveled.

In the first and third examples, the number of measurement periods Tm for acquiring the first or second value may be three or more, or four or more.

Extraction circuit 33 may extract at least one feature quantity using any of the methods of the first to third examples described above, or may extract a plurality of feature quantities using two or more of the methods of the first to third examples described above.

In the first to third examples described above, for example, extraction circuit 33 may extract one or more feature quantities based on the signal acquired during second and subsequent measurement periods Tm (i.e., measurement period Tm-2 and later) in the plurality of measurement periods Tm-1 to Tm-7 illustrated in FIGS. 9 and 10. As a result, the feature quantity is extracted using the signal that is output during second and subsequent measurement periods Tm when the drift is likely to be large. Therefore, the difference in the extracted feature quantity is likely to be large according to the type of the sample gas, thus enabling further improvement in identification accuracy. to be described later. When extraction circuit 33 extracts one or more feature quantities based on the signal acquired during second and subsequent measurement periods Tm, there may be measurement period Tm within second and subsequent measurement periods Tm in which extraction circuit 33 does not acquire the first value. That is, extraction circuit 33 may extract one or more feature quantities based on the signal acquired during any measurement period Tm among second and subsequent measurement periods Tm. For example, extraction circuit 33 may extract one or more feature quantities based on the signal acquired during third and subsequent measurement periods Tm in the plurality of measurement periods Tm-1 to Tm-7, or one or more feature quantities based on the signal acquired during fourth and subsequent measurement periods Tm in the plurality of measurement periods Tm-1 to Tm-7

Extraction circuit 33 may extract a feature quantity other than the feature quantity corresponding to the drift of the acquired signal. For example, extraction circuit 33 may acquire the signal values in a predetermined interval in at least one of second period T2 or third period T3 as a feature quantity. The predetermined interval is, for example, 0.1 second or more and 10 seconds or less.

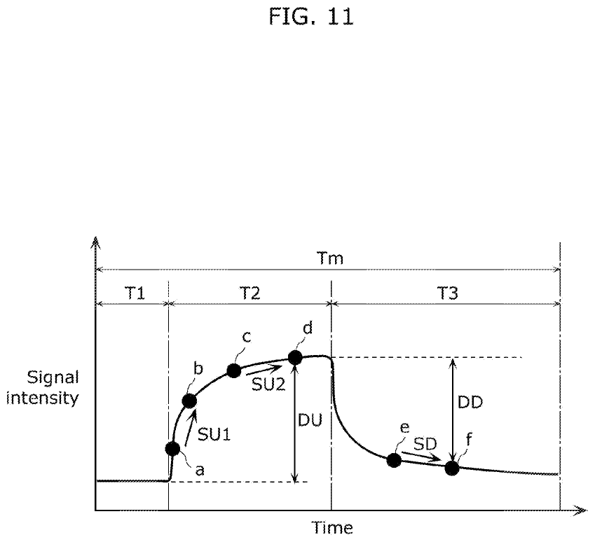

For example, in order to further improve the identification accuracy, extraction circuit 33 may acquire at least one of the rate of change or the amount of change in the signal value in at least one of second period T2 or third period T3 as a feature quantity in addition to the feature quantity corresponding to the drift. FIG. 11 is a diagram for explaining the rate of change (i.e., slope) and amount of change in the value of the signal output from sensor 10. FIG. 11 illustrates a temporal change in the intensity (e.g., voltage) of the signal output from sensor 10 acquired by acquisition circuit 32. In the graph in FIG. 11, each of points a to d is a point indicating the time and the signal value after the lapse of a predetermined time from the start of second period T2, and each of points e and f is a point indicating the time and the signal value after the lapse of a predetermined time from the start of third period T3. The number of points and the position of each point are set according to the type of sensor 10, the type of the identification target substance, and the like. The position of each point may be determined from the shape of the waveform of the signal instead of the predetermined time.

Extraction circuit 33 extracts, for example, a slope between two points in the graph of time and signal intensity as a feature quantity. The slopes extracted are, for example, slope SU1 of the line connecting points a and b in second period T2, slope SU2 of the line connecting points c and d in second period T2 later in time than point b, and slope SD of the line connecting points e and f in third period T3. Extraction circuit 33 also extracts the amount of change in a point from a predetermined time point in the graph of time and signal intensity as a feature quantity. The amounts of change extracted are, for example, the amount of change DU, which is the difference between the signal value at the start point of second period T2 and the signal value at point d, and amount of change DD, which is the difference between the signal value at the start point of third period T3 and the signal value at point f.

### (3) Identification Step

Referring again to FIG. 4, next, in the identification step, identification circuit 34 identifies the sample gas based on one or more feature quantities extracted in step S13, using a learned logical model for identifying the sample gas, and outputs the identification result (step S14). For example, identification circuit 34 receives one or more feature quantities as an input using the learned logical model, and outputs information indicating which of a plurality of identification target substances is contained in the sample gas. Identification circuit 34 may output information indicating whether or not the sample gas contains the identification target substance.

When the learned logical model includes a neural network, for example, one or more feature quantities are input to the input nodes of the neural network, and the probability of being contained in the sample gas for each of the plurality of identification target substances is output from the output node. That is, the number of input nodes is the number of one or more feature quantities that are input, and the number of output nodes is the number of a plurality of identification target substances to be identified. The learned logical model outputs the identification target substance with the highest probability of being output from the output node among the plurality of identification target substances. The learned logical model may output whether or not the sample gas contains the identification target substance based on the probability output from the output node, for example, based on whether or not the probability is greater than or equal to a threshold value.

As described above, the gas identification method performed by gas identification system 100 is a gas identification method using sensor 10, and includes: an acquisition step of acquiring a signal output from sensor 10 (step S12); an extraction step of extracting one or more feature quantities corresponding to the drift of the acquired signal (step S13); and an output step of identifying the sample gas based on the one or more feature quantities extracted, using a learned logical model, and outputting the identification result (step S14).

Thus, in the extraction step, one or more feature quantities corresponding to the drift of the signal are extracted, where the extracted feature quantity is different from the feature quantity corresponding to the output of the signal during the second period when sensor 10 is exposed to the sample gas, that is, the output depending on the adsorption concentration of the gas onto sensor 10. Therefore, in the identification step, identification based on the drift of the signal is performed, and even in the case of identifying sample gases with similar outputs from the sensor, which depend on the adsorption concentrations of the gases, the sample gases can be identified with high identification accuracy.

For example, in sensor 10, it is possible to accurately identify: a first sample gas for which the drift of the output signal is large, as in the signal illustrated in FIG. 6; and a second sample gas for which the waveform of the signal during the second period is the same as that for the first sample gas, but the drift of the signal hardly occurs, unlike the signal illustrated in FIG. 6.

### Example

Next, the present disclosure will be described in detail based on an example. However, the present disclosure is not limited in any way by the following example.

### [Acquisition of Signal for Identification Test]

First, using 16 sensors 10 disposed in housing 21, a signal output from each of 16 sensors 10 is acquired.

### <Sensor>

As each of the 16 sensors, sensor 10 with its sensing unit made of a different material was used. For example, sensor 10 with its sensing unit made of a resin material, such as methylphenyl silicone (a 75% phenyl group on the side chain) or methylphenyl silicone (a 35% phenyl group on the side chain), was used.

### <Gas>

As the reference gas, the air in a measurement chamber, where housing 21 was disposed, was used. Five types of sample gases A to E, obtained by volatilizing the following five types of chemical substances into the air in the measurement chamber, were used as the sample gases. That is, sample gases A to E contain the following corresponding chemical substances, respectively.
Sample gas A: Phenylethyl alcohol
Sample gas B: Methylcyclopentenolone
Sample gas C: Isovaleric acid
Sample gas D: Undecalactone
Sample gas E: Skatol

### <Acquisition of Voltage Signal>

The voltage signal acquisition operation started with the exposure of 16 sensors 10 to the reference gas for a first period of 5 seconds, followed by the exposure of 16 sensors 10 to the sample gas for a second period of 10 seconds and the exposure of 16 sensors 10 to the reference gas for a third period of 25 seconds. In a single voltage signal acquisition operation, the exposure operation in the measurement period made up of the first, second, and third periods was repeated seven times consecutively. As described above, in the single acquisition operation, 16 sensors 10 were exposed to the sample gas and the reference gas during seven consecutive measurement periods, and the voltage signal output from each of 16 sensors 10 was acquired. That is, through the single voltage signal acquisition operation, a voltage signal set made up of 16 patterns of voltage signals corresponding to 16 respective sensors 10 was acquired. The voltage signal set acquisition operation as described above was performed 158 times in total for the sample gases A to E. Specifically, the voltage signal set was acquired 30 times for sample gas A, and the voltage signal set was acquired 32 times for each of sample gases B to E.

### [Extraction of Feature Quantities]

A feature quantity was extracted from each of the total of 158 voltage signal sets corresponding to sample gases A to E, obtained by [Acquisition of Signal for Identification Test] described above.

### Reference Example

In extraction of feature quantities in a reference example, five feature quantities of slopes SU1, SU2, SD and amount of changes DU, DD illustrated in FIG. 11 were extracted from each of the 16 patterns of voltage signals that make up the voltage signal set. That is, for one voltage signal set, a feature quantity set made up of 80 feature quantities (16 patterns×5) was extracted. In this manner, a feature quantity set for each of the 158 voltage signal sets was extracted, and 158 feature quantity sets in the reference example were extracted.

### Example

In extraction of feature quantities in an example, a total of seven feature quantities were extracted from each of the 16 patterns of voltage signals that make up the voltage signal set. These seven feature quantities include the difference between values V1-7 and V1-2 and the difference between values V3-7 and V3-2 illustrated in FIG. 9, which are feature quantities corresponding to the drift of the signal, in addition to the same five feature quantities as in the reference example. That is, for one voltage signal set, a feature quantity set made up of 112 feature quantities (16 patterns×7) was extracted. In this manner, a feature quantity set for each of the 158 voltage signal sets was extracted, and 158 feature quantity sets in the example were extracted.

### [Identification Test]

When the five types of chemical substances described above were taken as identification target substances, a test was conducted to identify which of the five identification target substances was contained in the sample gas.

### <Distribution of Feature Quantity Sets>

The 158 feature quantity sets in each of the reference example and the example were randomly distributed by a computer into 128 feature quantity sets for training in each of the reference example and the examples and 32 feature quantity sets for prediction in each of the reference example and the examples, regardless of the type of the sample gas. Note that the distribution may be performed prior to the extraction of feature quantities described above.

### <Construction of Learned Logical Model>

A learned logical model for identifying an identification target substance was constructed by performing machine learning with a neural network that includes one hidden layer having five nodes, using, as teacher data, the 128 feature quantity sets for training in each of the reference example and the example and the identification target substances contained in the sample gases corresponding to the 128 feature quantity sets for training. The input of the neural network is the feature quantities that make up one feature quantity set, and the output of the neural network is the probability that each of the five identification target substances is contained in the sample gas. In the learned logical model, the identification target substance with the highest probability is identified as the identification target substance contained in the sample gas.

### <Identification of Sample Gas>

The identification target substance contained in the sample gas was identified using the learned logical model constructed as described above, with the feature quantities that make up the 32 feature quantity sets for prediction in each of the reference example and the example as inputs. Further, the distribution of the feature quantity set for training and the feature quantity set for prediction among the 158 sets was changed to reconstruct the learned logical model, and the identification of the identification target substance contained in the sample gas was performed three times in total.

Tables 1 to 3 show the results of the first to third times of identification using the feature quantity sets for prediction in the reference example. Tables 4 to 6 show the results of the first to third times of identification using the feature quantity sets for prediction in the example.

In each of Tables 1 to 6, the alphabets at the top and the alphabets at the leftmost correspond to sample gases A to E. Further, each cell represents the number of times the learned logical model identified the identification target substance contained in the sample gas listed at the leftmost of the row in which the cell is located, at the time of input of the feature quantities constituting the feature quantity set for prediction extracted from the signal corresponding to the sample gas among sample gases A to E listed at the top of the column in which the cell is located. That is, the numerical value in the cell for which the sample gases listed at the top and leftmost are the same is the number of times the identification was correct, and the numerical value in the cell for which the sample gases listed at the top and leftmost are different is the number of times the identification was incorrect.

TABLE 1

| Reference | | | | | |
|---|---|---|---|---|---|
| Example/1st time | A | B | C | D | E |
| A | 4 | 0 | 0 | 0 | 0 |
| B | 0 | 9 | 0 | 0 | 0 |
| C | 0 | 0 | 7 | 0 | 0 |
| D | 0 | 0 | 0 | 3 | 3 |
| E | 0 | 0 | 0 | 1 | 5 |

TABLE 2

| Reference | | | | | |
|---|---|---|---|---|---|
| Example/2nd time | A | B | C | D | E |
| A | 7 | 0 | 0 | 0 | 0 |
| B | 0 | 8 | 0 | 0 | 0 |
| C | 0 | 0 | 3 | 0 | 0 |
| D | 0 | 0 | 0 | 4 | 3 |
| E | 0 | 0 | 0 | 2 | 5 |

TABLE 3

| Reference | | | | | |
|---|---|---|---|---|---|
| Example/3rd time | A | B | C | D | E |
| A | 7 | 0 | 0 | 0 | 0 |
| B | 0 | 10 | 0 | 0 | 0 |
| C | 0 | 0 | 1 | 0 | 0 |
| D | 0 | 0 | 0 | 3 | 5 |
| E | 3 | 0 | 0 | 1 | 5 |

TABLE 4

| Example/1st time | A | B | C | D | E |
|---|---|---|---|---|---|
| A | 5 | 0 | 0 | 0 | 0 |
| B | 0 | 10 | 0 | 0 | 0 |
| C | 0 | 0 | 4 | 0 | 0 |
| D | 0 | 0 | 0 | 4 | 1 |
| E | 0 | 0 | 0 | 2 | 6 |

TABLE 5

| Example/2nd time | A | B | C | D | E |
|---|---|---|---|---|---|
| A | 6 | 0 | 0 | 0 | 0 |
| B | 0 | 9 | 0 | 0 | 0 |
| C | 0 | 0 | 5 | 0 | 0 |
| D | 0 | 0 | 0 | 7 | 0 |
| E | 0 | 0 | 0 | 1 | 4 |

TABLE 6

| Example/3rd time | A | B | C | D | E |
|---|---|---|---|---|---|
| A | 4 | 0 | 0 | 0 | 0 |
| B | 0 | 6 | 0 | 0 | 0 |
| C | 0 | 0 | 10 | 0 | 0 |
| D | 0 | 0 | 0 | 6 | 0 |
| E | 0 | 0 | 0 | 1 | 5 |

As shown in Tables 1 to 3, in the reference example, the number of times the identification was incorrect is high for the identification of sample gas D and sample gas E. Among the 32 feature quantity sets for prediction having been input, the percentages of the output of the incorrect identification result, that is, the percentages of false determination, were 12.5%, 15.6%, and 18.8% for the first to three times, respectively, and the average for the first to three times was 15.6%. Note that there was no false determination in the identification of sample gas A to sample gas C.

In contrast, as shown in Tables 4 to 6, in the example, the number of times the identification was incorrect is lower than in the reference example for the identifications of sample gas D and sample gas E. Among the 32 feature quantity sets for prediction having been input, the percentages of the output of the incorrect identification result, that is, the percentages of false determination, were 9.4%, 3.1%, and 3.1% for the first to three times, respectively, and the average for the first to three times was 5.2%. As described above, in the example, the percentage of false determination was 10% or more lower than in the reference example. Note that there was no false determination in the identification of sample gas A to sample gas C.

It can be seen from the above results that the percentage of false determination is lower and the identification accuracy is higher in the results of the example using the feature quantities corresponding to the drifts than in the results of the reference example not using the feature quantities corresponding to the drifts.

The waveform of the signal acquired from sensor 10 (for example, sensor 10 where the material of the sensing unit is made of methylphenyl silicone) was checked to find that the waveform of the signal during the second period varied among the case of exposure of sensor 10 to sample gas A, the case of exposure of sensor 10 to sample gas B, and the case of exposure of sensor 10 to sample gas C. Therefore, it is considered that the identification accuracy in each of sample gas A to sample gas C was high in both the reference example and the example.

On the other hand, the waveform of the signal during the second period was almost the same in the case of exposure of sensor 10 to sample gas D and in the case of exposure of sensor 10 to sample gas E. In addition, the drift of the signal corresponding to sample gas E was larger than the drift of the signal corresponding to sample gas D. Therefore, it is considered that false determination was more likely to occur especially in the reference example, and the identification accuracy was higher in the example using the feature quantities corresponding to the drifts.

### OTHER EMBODIMENTS

While the gas identification system and the gas identification method according to the present disclosure have been described above based on the embodiment and example, the disclosure is not limited to the embodiment and example. Forms obtained by applying various variations conceivable by a person skilled in the art to the embodiment and example, as well as other forms constructed by combining some components in the embodiment and example, are also within the scope of the present disclosure as long as those forms do not deviate from the spirit of the present disclosure.

In the above embodiment, exposure unit 20 has exposed sensor 10 to the reference gas during the first and third periods, but the present disclosure is not limited thereto. Exposure unit 20 simply needs to refrain from exposing sensor 10 to the sample gas during the first and third periods. For example, instead of exposing sensor 10 to the reference gas, exposure unit 20 may pull the sample gas and expose sensor 10 to the resultant vacuum atmosphere.

For example, gas identification system 100a has included detection device 200 and identification device 300, but the present disclosure is not limited thereto. Gas identification system 100a may include only identification device 300. In this case, for example, step S11 in FIG. 4 is omitted, and acquisition circuit 32a acquires, for example, the already detected sensor signal via the network.

For example, in the above embodiment, all or some of the components of the gas identification system according to the present disclosure may be formed of dedicated hardware, or may be implemented by executing a software program suitable for each component. Each component may be implemented by a program execution unit, such as a central processing unit (CPU) or processor, reading and executing a software program recorded in a recording medium, such as a hard disk drive (HDD) or a semiconductor memory.

The components of the gas identification system according to the present disclosure may include one or more electronic circuits. Each of the one or more electronic circuits may be a general-purpose circuit or a dedicated circuit.

One or more electronic circuits may include, for example, a semiconductor device, an integrated circuit (IC), a large-scale integrated circuit (LSI), or the like. The IC or LSI may be integrated on one chip or on a plurality of chips. The electronic circuit is referred to as the IC or LSI here, but the terminology used changes depending on the degree of integration, and the electronic circuit may be referred to as a system LSI, a very-large-scale integrated circuit (VLSI), or an ultra-large-scale integrated circuit (ULSI). A field-programmable gate array (FPGA) programmed after the manufacture of the LSI can also be used for the same purpose.

The general or specific aspect of the present disclosure may be realized by a system, device, method, integrated circuit, or computer program. The general or specific aspect of the present disclosure may also be realized by a computer-readable non-temporary recording medium such as an optical disk, HDD, or semiconductor memory in which the computer program is stored. The general or specific aspect of the present disclosure may also be realized by any combination of the system, device, method, integrated circuit, computer program, and recording medium.

For example, the present disclosure may be realized as a gas identification method to be executed by a computer, such as a gas identification system, or as a program to cause the computer to execute such a gas identification method. The present disclosure may be realized as a computer-readable non-transitory recording medium in which such a program is recorded.

### INDUSTRIAL APPLICABILITY

The gas identification system and gas identification method according to the present disclosure are useful for identifying a chemical substance and the like in gas.

1. A gas identification method using a sensor that outputs a signal corresponding to an adsorption concentration of a gas, the gas identification method comprising:
acquiring a signal output from the sensor that is exposed to a sample gas only during a second period out of a measurement period including a first period, the second period following the first period, and a third period following the second period;
extracting one or more feature quantities corresponding to a drift of the signal acquired; and
identifying the sample gas based on the one or more feature quantities extracted, using a learned logical model for identifying the sample gas, and outputting an identification result.

2. The gas identification method according to claim 1, further comprising:
exposing the sensor to the sample gas only during the second period out of the measurement period,
wherein in the acquiring, the signal output from the sensor exposed in the exposing is acquired.

3. The gas identification method according to claim 2,
wherein in the exposing, the sensor is exposed to a reference gas during each of the first period and the third period.

4. The gas identification method according to claim 1,
wherein in the acquiring, the signal output from the sensor is acquired via a network.

5. The gas identification method according to claim 1,
wherein in the extracting:
a first value is acquired, the first value being a value of the signal when the value of the signal which has changed due to exposure of the sensor to the sample gas during the second period is reverting to a reference value during the third period; and
at least one feature quantity among the one or more feature quantities is extracted by using the first value acquired.

6. The gas identification method according to claim 5,
wherein in the acquiring, the signal output from the sensor is acquired during a plurality of consecutive measurement periods each of which is the measurement period, and
in the extracting:
first values are acquired in two or more measurement periods among the plurality of consecutive measurement periods, the first values each being the first value; and
a difference between the first values acquired is extracted as the at least one feature quantity among the one or more feature quantities.

7. The gas identification method according to claim 5,
wherein in the acquiring, the signal output from the sensor is acquired during a plurality of consecutive measurement periods each of which is the measurement period, and
in the extracting:
first values are acquired in two or more measurement periods among the plurality of consecutive measurement periods, the first values each being the first value;
an approximate equation is derived by using the first values acquired; and
a coefficient of the approximate equation derived is extracted as the at least one feature quantity among the one or more feature quantities.

8. The gas identification method according to claim 5,
wherein in the extracting:
a second value is acquired, the second value being a value of the signal at an end of the first period; and
a difference between the first value acquired and the second value acquired is extracted as the at least one feature quantity among the one or more feature quantities.

9. The gas identification method according to claim 5,
wherein in the extracting, a value of the signal acquired at an end of the third period is acquired as the first value.

10. The gas identification method according to claim 1,
wherein in the acquiring, the signal output from the sensor is acquired during a plurality of consecutive measurement periods each of which is the measurement period, and
in the extracting:
third values are acquired in two or more measurement periods among the plurality of consecutive measurement periods, the third values each being a third value that is a value of the signal when the value of the signal changes due to exposure of the sensor to the sample gas during the second period; and
a difference between the third values acquired is extracted as at least one feature quantity among the one or more feature quantities.

11. The gas identification method according to claim 1,
wherein in the acquiring, the signal output from the sensor is acquired during a plurality of consecutive measurement periods each of which is the measurement period, and
in the extracting:
third values are acquired in two or more measurement periods among the plurality of consecutive measurement periods, the third values each being a third value that is a value of the signal when the value of the signal changes due to exposure of the sensor to the sample gas during the second period;
an approximate equation is derived by using the third values acquired; and
a coefficient of the approximate equation derived is extracted as at least one feature quantity among the one or more feature quantities.

12. The gas identification method according to claim 1,
wherein in the acquiring, the signal output from the sensor is acquired during a plurality of consecutive measurement periods each of which is the measurement period, and
in the extracting, at least one feature quantity among the one or more feature quantities is extracted based on the signal acquired during a second or subsequent measurement period in the plurality of measurement periods.

13. A gas identification system comprising:
a sensor that outputs a signal corresponding to an adsorption concentration of a gas;
an exposure unit that exposes the sensor to a sample gas only during a second period out of a measurement period including a first period, the second period following the first period, and a third period following the second period;
an acquisition circuit that acquires a signal output from the sensor in the measurement period;
an extraction circuit that extracts one or more feature quantities corresponding to a drift of the signal acquired,
a memory that stores a learned logical model for identifying the sample gas; and
an identification circuit that identifies the sample gas based on the one or more feature quantities extracted, using the learned logical model, and outputs an identification result.
